(19) United States
(12) Patent Application Publication
(10) Pub. No.: US 2010/0137327 A1
(43) Pub. Date: Jun. 3, 2010

(54) NOVEL SEMICARBAZIDE AND CARBONYLHYDRAZIDE DERIVATIVES USEFUL AS POTASSIUM CHANNEL MODULATORS

(75) Inventors: Antonio Nardi, Ballerup (DK); Joachim Demnitz, Kobenhavn (DK); Morten Grunnet, Kobenhavn (DK); Palle Christophersen, Ballerup (DK); David Spencer Jones, Smorum (DK); Elsebet Østergaard Nielsen, Kobenhavn (DK); Dorte Strøbæk, Farum (DK); Lars Siim Madsen, Soro (DK)

Correspondence Address: BIRCH STEWART KOLASCH & BIRCH, PO BOX 747, FALLS CHURCH, VA, 22040-0747, US

(73) Assignee: NeuroSearch A/S, Ballerup (DK)

(21) Appl. No.: 12/522,273

(22) PCT Filed: Jan. 17, 2008
(86) PCT No.: PCT/EP08/50487

§ 371 Date: Sep. 3, 2009

Related U.S. Application Data
(60) Provisional application No. 60/880,962, filed on Jan. 18, 2007.

(30) Foreign Application Priority Data
Jan. 18, 2007 (DK) PA 2007 00082

(51) Int. Cl. A61K 31/41; C07D 257/04; A61P 1/00; A61P 25/00; A61P 35/00; A61K 31/519; A61K 31/4985

(52) U.S. Cl. 514/250; 548/253; 514/381; 514/252.16

(57) ABSTRACT

This invention relates to novel semicarbazide and carbonylhydrazide derivatives that are found to be potent modulators of potassium channels and, as such, they are valuable candidates for the treatment of diseases or disorders as diverse as those which are responsive to modulation of potassium channels.

### TECHNICAL FIELD

This invention relates to novel semicarbazide and carbonylhydrazide derivatives that are found to be potent modulators of potassium channels and, as such, they are valuable candidates for the treatment of diseases or disorders as diverse as those which are responsive to modulation of potassium channels.

### BACKGROUND ART

Ion channels are cellular proteins that regulate the flow of ions through cellular membranes of all cells and are classified by their selective permeability to the different of ions (potassium, chloride, sodium etc.). Potassium channels, which represent the largest and most diverse sub-group of ion channels, selectively pass potassium ions and, doing so, they principally regulate the resting membrane potential of the cell and/or modulate their level of excitation.

Dysfunction of potassium channels, as well as other ion channels, generates loss of cellular control resulting in altered physiological functioning and disease conditions. Ion channel blockers and openers, by their ability to modulate ion channel function and/or regain ion channel activity in acquired or inherited channelopathies, are being used in the pharmacological treatment of a wide range of pathological diseases and have the potential to address an even wider variety of therapeutic indications. For instance, the primary indications for potassium channel openers encompass conditions as diverse as diabetes, arterial hypertension, cardiovascular diseases, urinary incontinence, atrial fibrillation, epilepsy, pain, and cancer.

Among the large number of potassium channel types, the large-conductance calcium-activated potassium channel subtype (BK) is an obvious site for pharmacological intervention and for the development of new potassium channel modulators. Their physiological role has been especially studied in the nervous system, where they are key regulators of neuronal excitability and of neurotransmitter release, and in smooth muscle, where they are crucial in modulating the tone of vascular, broncho-tracheal, urethral, uterine or gastro-intestinal musculature.

Given these implications, small agents with BK-opening properties could have a potentially powerful influence in the modulation and control of numerous consequences of muscular and neuronal hyperexcitability, such as asthma, urinary incontinence and bladder spasm, gastroenteric hypermotility, psychoses, post-stroke neuroprotection, convulsions, anxiety and pain. As far as the cardiovascular system is concerned, the physiological function of these ion channels represents a fundamental steady state mechanism, modulating vessel depolarisation, vasoconstriction and increases of intravascular pressure, and the development of selective activators of BK channels is seen as a potential pharmacotherapy of vascular diseases, including hypertension, erectile dysfunction, coronary diseases and vascular complications associated with diabetes or hypercholesterolemia.

### SUMMARY OF THE INVENTION

Is an object of the invention to provide novel semicarbazide and carbonylhydrazide derivatives useful as potassium channel modulators. The semicarbazide or carbonylhydrazide derivatives of the invention may be characterised by Formula I

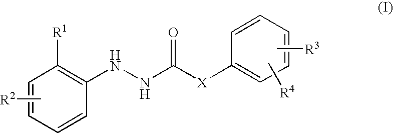

an enantiomer or a mixture of its enantiomers, or a pharmaceutically-acceptable addition salt thereof, wherein

X may be absent (representing a covalent bond) or may represent NH;

R¹ represents a tetrazolyl group;

R² represents halo, hydroxy or phenyl, which phenyl is optionally substituted one or more times with halo and/or trifluoromethyl; and

R³ and R⁴, independently of each other, represent halo, trifluoromethyl, hydroxy and/or phenyl.

In another aspect the invention provides pharmaceutical compositions comprising a therapeutically effective amount of the semicarbazide or carbonylhydrazide of the invention.

In a third aspect the invention relates to the use of the semicarbazide or carbonylhydrazide derivative of the invention for the manufacture of pharmaceutical compositions.

In a fourth aspect the invention provides a kit of parts comprising at least two separate unit dosage forms (A) and (B1) or (B2): (A) a semicarbazide or carbonylhydrazide derivative according to the invention; and (B1) a phosphodiesterase inhibitor, or (B2) an agent that potentiates endothelium-derived hyperpolarizing factor-mediated responses; and optionally (C) instructions for the simultaneous, sequential or separate administration of the semicarbazide or carbonylhydrazide derivative of A, and the phosphodiesterase inhibitor of B1, or an agent that potentiates endothelium-derived hyperpolarizing factor-mediated responses of B2, to a patient in need thereof.

In a further aspect the invention provides a method of treatment, prevention or alleviation of a disease or a disorder or a condition of a living animal body, including a human, which disorder, disease or condition is responsive to modulation of potassium channels, which method comprises the step of administering to such a living animal body in need thereof, a therapeutically effective amount of the semicarbazide or carbonylhydrazide derivative of the invention.

Other objects of the invention will be apparent to the person skilled in the art from the following detailed description and examples.

### DETAILED DISCLOSURE OF THE INVENTION

In its first aspect the invention provides novel semicarbazide and carbonylhydrazide derivatives of Formula I

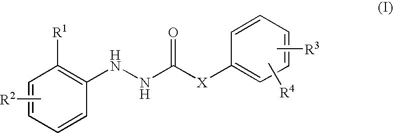

an enantiomer or a mixture of its enantiomers, or a pharmaceutically-acceptable addition salt thereof, wherein

X may be absent (representing a covalent bond) or may represent NH;

R¹ represents a tetrazolyl group;

R² represents halo, hydroxy or phenyl, which phenyl is optionally substituted one or more times with halo and/or trifluoromethyl; and

R³ and R⁴, independently of each other, represent halo, trifluoromethyl, hydroxy and/or phenyl.

In a preferred embodiment the semicarbazide or carbonylhydrazide derivative of the invention is a compound of Formula I, wherein X may be absent (representing a covalent bond) or may represent NH.

In a more preferred embodiment X is absent (representing a covalent bond).

In another more preferred embodiment X represents NH.

In another preferred embodiment the semicarbazide or carbonylhydrazide derivative of the invention is a compound of Formula I, wherein R¹ represents a tetrazolyl group.

In a more preferred embodiment R¹ represents a 1H-tetrazol-5-yl or 2H-tetrazol-5-yl group.

In a third preferred embodiment the semicarbazide or carbonylhydrazide derivative of the invention is a compound of Formula I, wherein R² represents halo, hydroxy or phenyl, which phenyl is optionally substituted one or more times with halo and/or trifluoromethyl.

In a more preferred embodiment R² represents phenyl, which phenyl is substituted one or more times with halo and/or trifluoromethyl.

In an even more preferred embodiment R² represents phenyl, which phenyl is substituted with halo and/or trifluoromethyl.

In a fourth preferred embodiment the semicarbazide or carbonylhydrazide derivative of the invention is a compound of Formula I, wherein R³ and R⁴, independently of each other, represent halo, trifluoromethyl, hydroxy and/or phenyl.

In a more preferred embodiment R³ and R⁴ both represent halo or trifluoromethyl.

In a most preferred embodiment the semicarbazide or carbonylhydrazide derivative of the invention is

N-[3,5-bis(trifluoromethyl)phenyl]-2-[3-(1H-tetrazol-5-yl)-4′-(trifluoromethyl)-[1,1′-biphenyl]-4-yl]-hydrazine carboxamide; or

3,5-Bis-trifluoromethyl-benzoic acid N′-[3-(1H-tetrazol-5-yl)-4′-trifluoro-methyl-biphenyl-4-yl]-hydrazide;

or a pharmaceutically-acceptable addition salt thereof.

Any combination of two or more of the embodiments described herein is considered within the scope of the present invention.

### Definition of Substituents

In the context of this invention halo represents fluoro, chloro, bromo or iodo.

### Pharmaceutically Acceptable Salts

The semicarbazide or carbonylhydrazide derivatives of the invention may be provided in any form suitable for the intended administration. Suitable forms include pharmaceutically (i.e. physiologically) acceptable salts, and pre- or prodrug forms of the compound of the invention.

Examples of pharmaceutically acceptable addition salts include, without limitation, the non-toxic inorganic and organic acid addition salts such as the hydro-chloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a semicarbazide or carbonylhydrazide derivative of the invention include, without limitation, the sodium, the potassium, the calcium, the magnesium, the lithium, and the ammonium salt, and the like, of a compound of the invention containing an anionic group. Such cationic salts may be formed by procedures well known and described in the art.

### Methods of Preparation

The semicarbazide or carbonylhydrazide derivatives of the invention may be prepared by conventional methods for chemical synthesis, e.g. those described in the working examples.

More generally, the procedure is outlined in Scheme 1 below. INT-1, which commercially-available or easily prepared by conventional synthetic methods, is converted into the hydrazine derivative (INT-2) upon treatment with sulphuric acid 50%, sodium nitrite and tin(II) chloride. The hydrazine derivative (INT-2), in turn, is reacted with commercially-available and properly-substituted acid chlorides and isocyanates, to afford the correspondent carbonylhydrazide derivatives I and semicarbazide derivatives II, respectively.

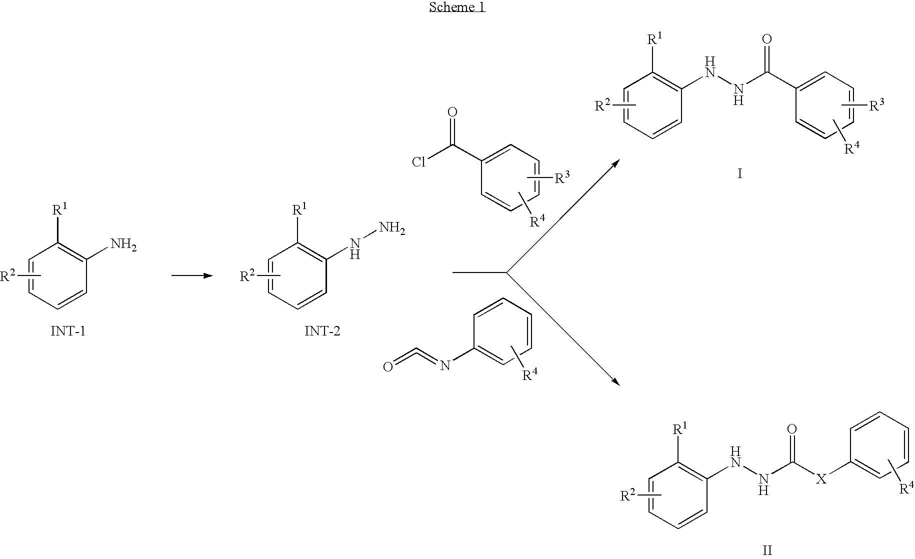

### Biological Activity

The semicarbazide or carbonylhydrazide derivatives of the invention have been found to possess potassium channel modulating activity as measured by standard electrophysiological methods, and are thus believed to belong to a new chemical class of potassium channel modulators. Due to their activity at the potassium channels, the compounds of the invention are considered useful for the treatment of a wide range of diseases and conditions.

In a special embodiment, the semicarbazide or carbonylhydrazide derivatives of the invention are considered useful for the treatment, prevention or alleviation of a respiratory disease, epilepsy, convulsions, seizures, absence seizures, vascular spasms, coronary artery spasms, motor neuron diseases, myokymia, renal disorders, polycystic kidney disease, bladder hyperexcitability, bladder spasms, urinogenital disorders, urinary incontinence, bladder outflow obstruction, erectile dysfunction, gastrointestinal dysfunction, gastrointestinal hypomotility disorders, gastrointestinal motility insufficiency, postoperative ileus, constipation, gastro-esophageal reflux disorder, secretory diarrhoea, an obstructive or inflammatory airway disease, ischaemia, cerebral ischaemia, ischaemic heart disease, angina pectoris, coronary heart disease, ataxia, traumatic brain injury, stroke, Parkinson's disease, bipolar disorder, psychosis, schizophrenia, autism, anxiety, mood disorders, depression, manic depression, psychotic disorders, dementia, learning deficiencies, age related memory loss, memory and attention deficits, Alzheimer's disease, amyotrophic lateral sclerosis (ALS), dysmenorrhoea, narcolepsy, sleeping disorders, sleep apnoea, Reynaud's disease, intermittent claudication, Sjögren's syndrome, xerostomia, arrhythmia, cardiovascular disorders, hypertension, myotonic dystrophy, myotonic muscle dystrophia, spasticity, xerostomia, diabetes Type II, hyperinsulinemia, premature labour, cancer, brain tumours, inflammatory bowel disease, irritable bowel syndrome, colitis, colitis Crohn, immune suppression, hearing loss, migraine, pain, neuropathic pain, inflammatory pain, trigeminal neuralgia, vision loss, rhinorrhoea, ocular hypertension (glaucoma), baldness, cardiac arrhythmia, atrial arrhythmia, ventricular arrhythmia, atrial fibrillation, ventricular fibrillation, tachyarrhythmia, atrial tachyarrhythmia, ventricular tachyarrhythmia, bradyarrhythmia, or any other abnormal rhythm, e.g. caused by myocardial ischaemia, myocardial infarction, cardiac hypertrophy or cardiomyopathy.

In a more preferred embodiment, the semicarbazide or carbonylhydrazide derivatives of the invention are considered useful for the treatment, prevention or alleviation of a respiratory disease, urinary incontinence, erectile dysfunction, anxiety, epilepsy, psychosis, schizophrenia, bipolar disorder, depression, amyotrophic lateral sclerosis (ALS), Parkinson's disease or pain.

In another more preferred embodiment, the semicarbazide or carbonylhydrazide derivatives of the invention are considered useful for the treatment, prevention or alleviation of psychosis, schizophrenia, bipolar disorder, depression, epilepsy, Parkinson's disease or pain.

In a third more preferred embodiment, the semicarbazide or carbonylhydrazide derivatives of the invention are considered useful for the treatment, prevention or alleviation of pain, mild or moderate or severe pain, pain of acute, chronic or recurrent character, pain caused by migraine, postoperative pain, phantom limb pain, inflammatory pain, neuropathic pain, chronic headache, central pain, pain related to diabetic neuropathy, to post therapeutic neuralgia, or to peripheral nerve injury.

In a fourth more preferred embodiment, the semicarbazide or carbonylhydrazide derivatives of the invention are considered useful for the treatment, prevention or alleviation of cardiac arrhythmia, atrial arrhythmia, ventricular arrhythmia, atrial fibrillation, ventricular fibrillation, tachyarrhythmia, atrial tachyarrhythmia, ventricular tachyarrhythmia, bradyarrhythmia, or any other abnormal rhythm, e.g. caused by myocardial ischaemia, myocardial infarction, cardiac hypertrophy, cardiomyopathy or a genetic disease.

In a fifth more preferred embodiment, the semicarbazide or carbonylhydrazide derivatives of the invention are considered useful for the treatment, prevention or alleviation of cardiac ischemia, ischemic heart disease, hypertrophic heart, cardiomyopathy or failing heart.

In a sixth more preferred embodiment, the semicarbazide or carbonylhydrazide derivative of the invention are considered useful for the treatment, prevention or alleviation of a cardiovascular disease. In a more preferred embodiment the cardiovascular disease is atherosclerosis, ischemia/reperfusion, hypertension, restenosis, arterial inflammation, myocardial ischaemia or ischaemic heart disease.

In a seventh more preferred embodiment, the semicarbazide or carbonylhydrazide derivative of the invention are considered useful for the treatment, prevention or alleviation of cardiac arrhythmia, atrial fibrillation and/or ventricular tachyarrhythmia.

In an eighth more preferred embodiment, the semicarbazide or carbonylhydrazide derivative of the invention are considered useful for obtaining preconditioning of the heart. Preconditioning, which includes ischemic preconditioning and myocardial preconditioning, describes short periods of ischemic events before initiation of a long lasting ischemia. The semicarbazide or carbonylhydrazide derivatives of the invention are believed having an effect similar to preconditioning obtained by such ischemic events. Preconditioning protects against later tissue damage resulting from the long lasting ischemic events.

In an eight more preferred embodiment, the semicarbazide or carbonylhydrazide derivative of the invention are considered useful for the treatment, prevention or alleviation of schizophrenia, depression or Parkinson's disease.

In a ninth more preferred embodiment, the semicarbazide or carbonylhydrazide derivative of the invention are considered useful for the treatment, prevention or alleviation of an obstructive or inflammatory airway disease. In a more preferred embodiment the the obstructive or inflammatory airway disease is an airway hyperreactivity, a pneumoconiosis such as aluminosis, anthracosis, asbestosis, chalicosis, ptilosis, siderosis, silicosis, tabacosis and byssinosis, a chronic obstructive pulmonary disease (COPD), bronchitis, excerbation of airways hyperreactivity or cystic fibrosis.

In its most preferred embodiment the obstructive airway disease is chronic obstructive pulmonary disease (COPD).

In a tenth more preferred embodiment, the semicarbazide or carbonylhydrazide derivative of the invention are considered useful for the treatment, prevention or alleviation of a sexual dysfunction, incl. male sexual dysfunction and female sexual dysfunction, and incl. male erectile dysfunction.

In an even more preferred embodiment the semicarbazide or carbonylhydrazide derivative of the invention may be co-administered with a phosphodiesterase inhibitor, in particular a phosphodiesterase 5 (PDE5) inhibitor, e.g. sildenafil, tadalafil, vardenafil and dipyridamole, or with an agent that potentiates endothelium-derived hyperpolarizing factor-mediated responses, in particular calcium dobesilate or similar 2,5-dihydroxybenzenesulfonate analogs.

In a most preferred embodiment the semicarbazide or carbonylhydrazide derivatives of the invention is used in a combination therapy together with sildenafil, tadalafil, vardenafil or calcium dobesilate.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

Preferred compounds of the invention show a biological activity in the sub-micromolar and micromolar range, i.e. of from below 1 to about 100 μM.

### Pharmaceutical Compositions

In another aspect the invention provides novel pharmaceutical compositions comprising a therapeutically effective amount of a semicarbazide or carbonylhydrazide derivative of the invention.

While a semicarbazide or carbonylhydrazide derivative of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the semicarbazide or carbonylhydrazide derivative of the invention together with one or more pharmaceutically acceptable carriers therefore, and, optionally, other therapeutic and/or prophylactic ingredients know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by any person skilled in the art, by use of standard methods and conventional techniques, appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Pharmaceutical Kits of Parts

According to the invention there is also provided a kit of parts comprising at least two separate unit dosage forms (A) and (B):

(A) a semicarbazide or carbonylhydrazide derivative of the invention; and

(B1) a phosphodiesterase inhibitor, or

(B2) an agent that potentiates endothelium-derived hyperpolarizing factor-mediated responses; and optionally

(C) instructions for the simultaneous, sequential or separate administration of the semicarbazide or carbonylhydrazide derivative of A, and the phosphodiesterase inhibitor of B1, or an agent that potentiates endothelium-derived hyperpolarizing factor-mediated responses of B2, to a patient in need thereof.

In a more preferred embodiment the phosphodiesterase inhibitor for use according to the invention (B1) is a phosphodiesterase 5 (PDE5) inhibitor, and in an even more preferred embodiment the phosphodiesterase inhibitor for use according to the invention is sildenafil, tadalafil or vardenafil.

In another more preferred embodiment the agent that potentiates endothelium-derived hyperpolarizing factor-mediated responses for use according to the invention (B2) is calcium dobesilate.

The semicarbazide or carbonylhydrazide derivative of the invention and the phosphodiesterase inhibitor or the agent that potentiates endothelium-derived hyperpolarizing factor-mediated responses for use according to the invention may preferably be provided in a form that is suitable for administration in conjunction with the other. This is intended to include instances where one or the other of two formulations may be administered (optionally repeatedly) prior to, after, and/or at the same time as administration with the other component.

Also, the semicarbazide or carbonylhydrazide derivative of the invention and the phosphodiesterase inhibitor or the agent that potentiates endothelium-derived hyperpolarizing factor-mediated responses for use according to the invention may be administered in a combined form, or separately or separately and sequentially, wherein the sequential administration is close in time or remote in time. This may in particular include that two formulations are administered (optionally repeatedly) sufficiently closely in time for there to be a beneficial effect for the patient, that is greater over the course of the treatment of the relevant condition than if either of the two formulations are administered (optionally repeatedly) alone, in the absence of the other formulation, over the same course of treatment. Determination of whether a combination provides a greater beneficial effect in respect of, and over the course of treatment of, a particular condition, will depend upon the condition to be treated or prevented, but may be achieved routinely by the person skilled in the art.

When used in this context, the terms “administered simultaneously” and “administered at the same time as” include that individual doses of the positive allosteric nicotine receptor modulator and the cognitive enhancer are administered within 48 hours, e.g. 24 hours, of each other.

Bringing the two components into association with each other, includes that components (A) and (B) may be provided as separate formulations (i.e. independently of one another), which are subsequently brought together for use in conjunction with each other in combination therapy; or packaged and presented together as separate components of a “combination pack” for use in conjunction with each other in combination therapy.

### Methods of Therapy

In another aspect the invention provides a method of treatment, prevention or alleviation of a disease or a disorder or a condition of a living animal body, including a human, which disorder, disease or condition is responsive to modulation of potassium channels, which method comprises the step of administering to such a living animal body in need thereof, a therapeutically effective amount of the semicarbazide or carbonylhydrazide derivative of the invention.

The preferred medical indications contemplated according to the invention are those stated above.

It is at present contemplated that a suitable dosage of the active pharmaceutical ingredient (API) is within the range of from about 0.1 to about 1000 mg API per day, more preferred of from about 1 to about 500 mg API per day, most preferred of from about 1 to about 100 mg API per day, dependent, however, upon the exact mode of administration, the form in which it is administered, the indication considered, the subject and in particular the body weight of the subject involved, and further the preference and experience of the physician or veterinarian in charge.

### BRIEF DESCRIPTION OF THE DRAWING

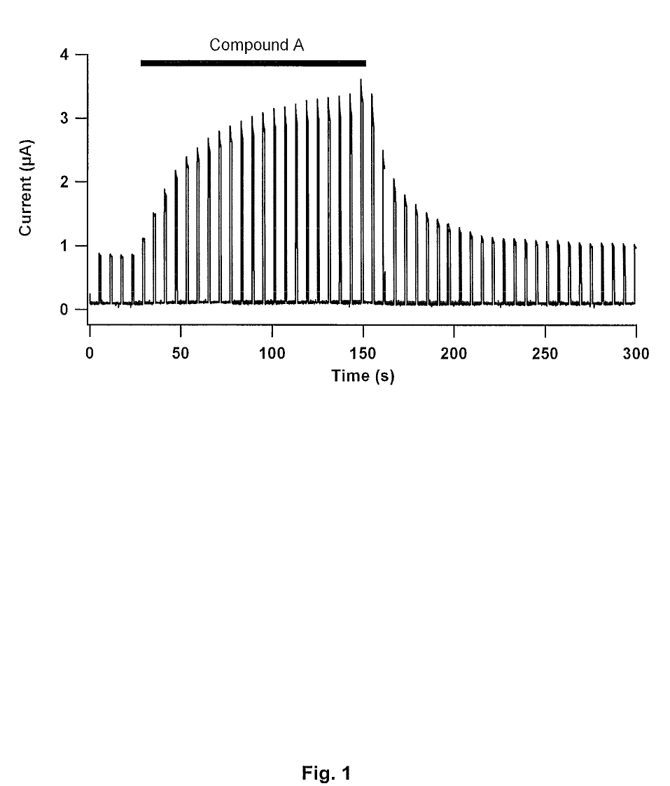

The present invention is further illustrated by reference to the accompanying drawing, in which FIG. 1 shows the BK channel opening activity [current (μA) vs. time (s)] of a carbonylhydrazide derivative representative of the invention, i.e. Compound 2, determined by a standard electrophysiological method using BK channels heterologously expressed in Xenopus laevis oocytes.

### EXAMPLES

The invention is further illustrated with reference to the following examples, which are not intended to be in any way limiting to the scope of the invention as claimed.

### Example 1

### Preparatory Example

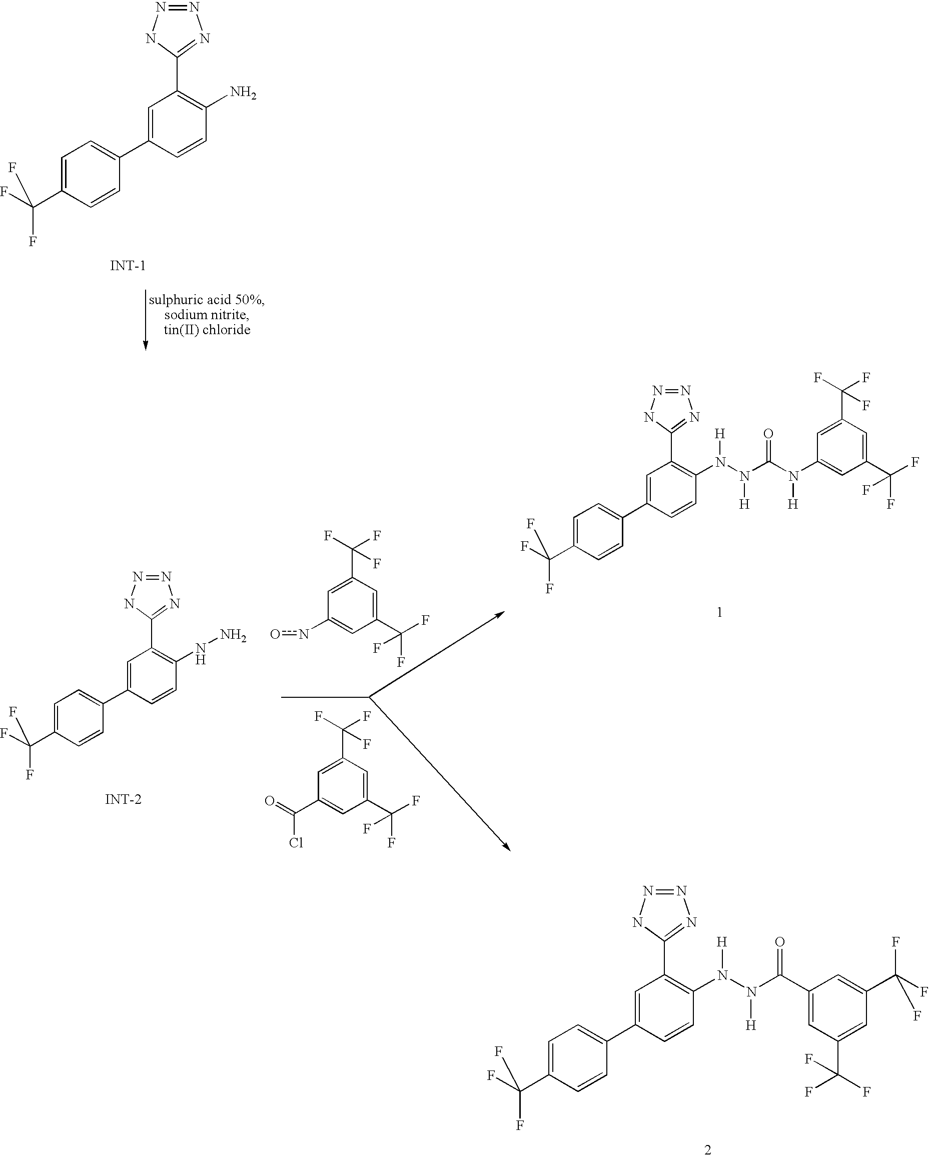 [0086] [3-(1H-Tetrazol-5-yl)-4′-trifluoromethyl-biphenyl-4-yl]-hydrazine (INT-2)

To an aged (30 min), ice-cooled and vigorously stirred suspension of INT-1 (1 g, 3.28 mmol) (prepared as described in WO 2004/111017) in H₂SO₄ 50% (100 ml), a solution of sodium nitrite (0.27 g, 3.94 mmol) in water (4 ml) is added drop wise. After the addition, stirring and icing is continued for further 30 min and a solution of tin(II) chloride (1.36 g, 7.2 mmol) in 3 ml of concentrated HCl is then added dropwise. The reaction mixture was kept stirring for an additional hour at 0° C. and the resulting white precipitate is collected by filtration. The product is dried in vacuo to yield 1.372 g of hydrazine derivative as a sulphate salt (LC-MS, basic conditions, one peak, r.t. 1.35 min, M-319).
N-[3,5-bis(trifluoromethyl)phenyl]-2-[3-(1H-tetrazol-5-yl)-4′-(trifluoromethyl)[1,1′-biphenyl]-4-yl]-hydrazine carboxamide (Compound 1)

To a mixture of INT-1 (0.686 g, 1.64 mmol of free hydrazine derivative) in pyridine (5 ml), 3,5-bis(trifluoromethyl)phenylisocyanate is added drop wise (0.418 g, 1.64 mmol) and the resulting reaction mixture is first heated (50° C.) for 5 hours and then evaporated to dryness (0.94 g, yield ˜100%), to afford Compound 1. LC-MS, basic conditions, r.t 2.02 min, >95%. Purification has been carried out by prep LC-MS. LC-ESI-HRMS of [M—H]— shows 574.1052 Da. Calc. 574.103785 Da, dev. 2.5 ppm.
3,5-Bis-trifluoromethyl-benzoic acid N40 -[3-(1H-tetrazol-5-yl)-4′-trifluoromethyl-biphenyl-4-yl]-hydrazide (Compound 2)

To a mixture of (0.686 g, 1.64 mmol of free hydrazine derivative) in pyridine (5 ml), 3,5-bis(trifluoromethyl)benzoylchloride (0.453 g, 1.64 mmol) is added drop wise and the resulting reaction mixture is first heated (50° C.) for 5 hours and then evaporated to dryness (0.92 g, yield ˜100%), to afford Compound 2. LC-MS, basic conditions, r.t 2.03 min, >90%. Purification has been carried out by prep LC-MS. LC-ESI-HRMS of [M—H]— shows 559.09 Da. Calc. 559.092886 Da, dev. −5.2 ppm.

### Example 2

### Biological Activity

### Expression and Functional Characterization of the BK Channel

In this example the BK channel opening activity of a carbonylhydrazide derivative for use according to the invention, Compound 2 (herein designated Compound A), is determined using BK channels heterologously expressed in Xenopus laevis oocytes.

The electrical current through the BK channel is measured conventional two-electrode voltage clamp. BK current is activated by repeated step protocols. In brief, this protocol goes from a resting membrane potential of −40 mV lasting for 5 s to a depolarised step to +30 mV lasting for 1 s. The protocol was repeated continuously.

Having reached a stable current level, Compound A (30 μM), was added. A marked increase in the current activated by depolarisation could be observed. The results are presented in FIG. 1.

1-16. (canceled)

17. A semicarbazide or carbonylhydrazide derivative of Formula I 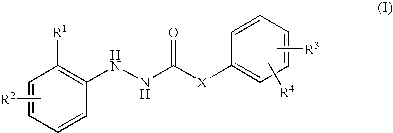
an enantiomer or a mixture of its enantiomers, or a pharmaceutically-acceptable addition salt thereof wherein
X may be absent (representing a covalent bond) or may represent NH;
R¹ represents a tetrazolyl group;
R² represents halo, hydroxy or phenyl, which phenyl is optionally substituted one or more times with halo and/or trifluoromethyl; and
R³ and R⁴, independently of each other, represent halo, trifluoromethyl, hydroxy and/or phenyl.

18. The semicarbazide or carbonylhydrazide derivative of claim 17, an enantiomer or a mixture of its enantiomers, or a pharmaceutically-acceptable addition salt thereof, wherein X may be absent (representing a covalent bond) or may represent NH.

19. The semicarbazide or carbonylhydrazide derivative of claim 17, an enantiomer or a mixture of its enantiomers, or a pharmaceutically-acceptable addition salt thereof, wherein R¹ represents a tetrazolyl group.

20. The semicarbazide or carbonylhydrazide derivative of claim 17, an enantiomer or a mixture of its enantiomers, or a pharmaceutically-acceptable addition salt thereof, wherein R² represents halo, hydroxy or phenyl, which phenyl is optionally substituted one or more times with halo and/or trifluoromethyl.

21. The semicarbazide or carbonylhydrazide derivative of claim 17, an enantiomer or a mixture of its enantiomers, or a pharmaceutically-acceptable addition salt thereof, wherein R³ and R⁴, independently of each other, represent halo, trifluoromethyl, hydroxy and/or phenyl.

22. The semicarbazide or carbonylhydrazide derivative of claim 17, which is
N-[3,5-bis(trifluoromethyl)phenyl]-2-[3-(1H-tetrazol-5-yl)-4′-(trifluoromethyl)[1,1′-biphenyl]-4-yl]-hydrazine carboxamide; or
3,5-Bis-trifluoromethyl-benzoic acid N′-[3-(1H-tetrazol-5-yl)-4′-trifluoromethyl-biphenyl-4-yl]-hydrazide;
an enantiomer or a mixture of its enantiomers, or a pharmaceutically-acceptable addition salt thereof.

23. A pharmaceutical composition comprising a therapeutically effective amount of the semicarbazide or carbonylhydrazide derivative of claim 17, an enantiomer or a mixture of its enantiomers, or a pharmaceutically-acceptable addition salt thereof, together with one or more adjuvants, excipients, carriers and/or diluents.

24. A method of treatment, prevention or alleviation of a disease or a disorder or a condition of a living animal body, including a human, which disorder, disease or condition is responsive to modulation of potassium channels, which method comprises the step of administering to such a living animal body in need thereof, a therapeutically effective amount of the semicarbazide or carbonylhydrazide derivative according to claim 17, an enantiomer or a mixture of its enantiomers, or a pharmaceutically-acceptable addition salt thereof.

25. The method according to claim 24, wherein the disease, disorder or condition is a respiratory disease, epilepsy, convulsions, seizures, absence seizures, vascular spasms, coronary artery spasms, motor neuron diseases, myokymia, renal disorders, polycystic kidney disease, bladder hyperexcitability, bladder spasms, urinogenital disorders, urinary incontinence, bladder outflow obstruction, erectile dysfunction, gastrointestinal dysfunction, gastrointestinal hypomotility disorders, gastrointestinal motility insufficiency, postoperative ileus, constipation, gastroesophageal reflux disorder, secretory diarrhoea, an obstructive or inflammatory airway disease, ischaemia, cerebral ischaemia, ischaemic heart disease, angina pectoris, coronary heart disease, ataxia, traumatic brain injury, stroke, Parkinson's disease, bipolar disorder, psychosis, schizophrenia, autism, anxiety, mood disorders, depression, manic depression, psychotic disorders, dementia, learning deficiencies, age related memory loss, memory and attention deficits, Alzheimer's disease, amyotrophic lateral sclerosis (ALS), dysmenorrhea, narcolepsy, sleeping disorders, sleep apnea, Reynaud's disease, intermittent claudication, Sjögren's syndrome, xerostomia, arrhythmia, cardiovascular disorders, hypertension, myotonic dystrophy, myotonic muscle dystrophia, spasticity, xerostomi, diabetes Type II, hyperinsulinemia, premature labour, cancer, brain tumors, inflammatory bowel disease, irritable bowel syndrome, colitis, colitis Crohn, immune suppression, hearing loss, migraine, pain, neuropathic pain, inflammatory pain, trigeminal neuralgia, vision loss, rhinorrhoea, ocular hypertension (glaucoma), baldness, cardiac arrhythmia, atrial arrhythmia, ventricular arrhythmia, atrial fibrillation, ventricular fibrillation, tachyarrhythmia, atrial tachyarrhythmia, ventricular tachyarrhythmia, bradyarrhythmia, or any other abnormal rhythm, e.g. caused by myocardial ischaemia, myocardial infarction, cardiac hypertrophy or cardiomyopathy.

26. A kit of parts comprising at least two separate unit dosage forms (A) and (B1) or (B2):
(A) a semicarbazide or carbonylhydrazide derivative according to claim 17, or an enantiomer or a mixture of its enantiomers, or pharmaceutically-acceptable addition salts thereof; and
(B1) a phosphodiesterase inhibitor; or
(B2) an agent that potentiates endothelium-derived hyperpolarizing factor-mediated responses; and optionally
(C) instructions for the simultaneous, sequential or separate administration of the semicarbazide or carbonylhydrazide derivative of A, and the phosphodiesterase inhibitor of B1, or an agent that potentiates endothelium-derived hyperpolarizing factor-mediated responses of B2, to a patient in need thereof.

27. A method of treatment or alleviation of a sexual dysfunction, which method comprises the step of administering to such a living animal body in need thereof, a therapeutically effective amount of a combination of p1 (A) a semicarbazide or carbonylhydrazide derivative according to claims 17; and
(B1) a phosphodiesterase inhibitor; or
(B2) an agent that potentiates endothelium-derived hyperpolarizing factor-mediated responses;
an enantiomer or a mixture of its enantiomers, or pharmaceutically-acceptable addition salts thereof.

28. The method of claim 27, wherein the sexual dysfunction is a male sexual dysfunction, a female sexual dysfunction or a male erectile dysfunction.

29. The method according to claim 27, wherein the phosphordiesterase inhibitor of is sildenafil, tadalafil or vardenafil; and the agent that potentiates endothelium-derived hyperpolarizing factor-mediated responses is calcium dobesilate.
